(19) United States
(12) Patent Application Publication
(10) Pub. No.: US 2026/0027969 A1
(43) Pub. Date: Jan. 29, 2026

(54) NOTIFICATION APPARATUS, NOTIFICATION METHOD, AND RECORDING MEDIUM

(71) Applicant: HONDA MOTOR CO., LTD., Tokyo (JP)

(72) Inventors: Satoru Shinkawa, Wako-shi (JP); Shigenobu Mitsuzawa, Wako-shi (JP); Hiroshi Ono, Wako-shi (JP); Hiroki Taniguchi, Wako-shi (JP); Nozomi Nagatani, Wako-shi (JP)

(21) Appl. No.: 19/211,878

(22) Filed: May 19, 2025

(30) Foreign Application Priority Data
Jul. 26, 2024 (JP) 2024-121022

(51) Int. Cl. B60Q 9/00; A61B 5/00; A61B 5/18; G10L 15/22; G10L 25/66

(52) U.S. Cl. CPC B60Q 9/00; A61B 5/18; A61B 5/4803; G10L 15/22; G10L 25/66

(57) ABSTRACT

A communication terminal functioning as a notification apparatus includes: an information acquisition unit acquiring biological information about a driver being a user at time intervals; an intellectual function evaluation unit evaluating a first physical condition indicating a relatively long-term physical condition, based on a plurality of pieces of the biological information of a predetermined period acquired by the information acquisition unit; a dialog unit performing speech output encouraging a speech response from the driver, when that the driver has gotten in a vehicle is detected; a speech evaluation unit evaluating a second physical condition indicating a relatively short-term physical condition, based on speech information of the driver as a response to the speech output; and a notification control unit notifying the driver of notification information corresponding to a physical condition, based on a combination of evaluation results from the intellectual function evaluation unit and from the speech evaluation unit.

### INCORPORATION BY REFERENCE

The present application claims priority under 35 U.S.C. § 119 to Japanese Patent Application No. 2024-121022 filed on Jul. 26, 2024. The content of the application is incorporated herein by reference in its entirety.

### BACKGROUND OF THE INVENTION

### Field of the Invention

The present invention relates to a notification apparatus, a notification method, and a recording medium.

### Description of the Related Art

In recent years, efforts have gained momentum for providing access to sustainable transport systems that also take into account people in vulnerable situations among traffic participants. Towards this end, efforts are focused on research and development for further improving traffic safety, convenience, and the like through research and development relating to a preventive safety technology. For example, Japanese Patent Laid-Open No. 2019-200544 and Japanese Patent Laid-Open No. 2023-145868 disclose a driving assistance apparatus that estimates a cognitive state of a driver using vehicle information and biological information consisting of an imaging result, a respiration, and a pulse wave of the driver, or that gives advice, a warning, or the like to the driver.

However, in the preventive safety technology, it is desired to take necessary response measures in accordance with a physical condition of the driver that affects a cognitive ability, a judgment ability, and the like of the driver. As one of the necessary response measures, a problem is to detect the physical condition of a user with high accuracy and to provide notification in accordance with the detected physical condition.

In order to solve the above problem, the present application has an object to separate chronic physical conditions from temporary physical conditions and provide appropriate notification in accordance with the physical condition. Thus, the present application further improves traffic safety and contributes to development of a sustainable transport system.

### SUMMARY OF THE INVENTION

According to an aspect of the present disclosure, a notification apparatus includes: a biological information acquisition unit configured to acquire biological information about a user at time intervals; a first physical condition evaluation unit configured to evaluate a first physical condition indicating a relatively long-term physical condition of the user, based on a plurality of pieces of the biological information of a predetermined period acquired by the biological information acquisition unit; a speech output unit configured to perform speech output encouraging a speech response from the user, when detecting that the user has gotten in a target vehicle; a second physical condition evaluation unit configured to evaluate a second physical condition indicating a relatively short-term physical condition of the user, based on speech information of the user as a response to the speech output; and a notification control unit configured to notify the user of notification information corresponding to a physical condition of the user, based on a combination of an evaluation result from the first physical condition evaluation unit and an evaluation result from the second physical condition evaluation unit.

According to another aspect of the present disclosure, a notification method executed by a notification apparatus includes: a biological information acquisition step of acquiring biological information about a user at time intervals; a first physical condition evaluation step of evaluating a first physical condition indicating a relatively long-term physical condition of the user, based on a plurality of pieces of the biological information of a predetermined period acquired in the biological information acquisition step; a speech output step of performing speech output encouraging a speech response from the user, when detecting that the user has gotten in a target vehicle; a second physical condition evaluation step of evaluating a second physical condition indicating a relatively short-term physical condition of the user, based on speech information of the user corresponding to the speech output; and a notification control step of notifying the user of notification information corresponding to a physical condition of the user, based on a combination of an evaluation result from the first physical condition evaluation step and an evaluation result from the second physical condition evaluation step.

According to another aspect of the present disclosure, a non-transitory computer readable recording medium recording a program causes a notification apparatus controlled by a computer to function as: a biological information acquisition unit configured to acquire biological information about a user at time intervals; a first physical condition evaluation unit configured to evaluate a first physical condition indicating a relatively long-term physical condition of the user, based on a plurality of pieces of the biological information of a predetermined period acquired by the biological information acquisition unit; a speech output unit configured to perform speech output encouraging a speech response from the user, when detecting that the user has gotten in a target vehicle; a second physical condition evaluation unit configured to evaluate a second physical condition indicating a relatively short-term physical condition of the user, based on speech information of the user as a response to the speech output; and a notification control unit configured to notify the user of notification information corresponding to a physical condition of the user, based on a combination of an evaluation result from the first physical condition evaluation unit and an evaluation result from the second physical condition evaluation unit.

According to the present invention, it is possible to separate chronic physical conditions from temporary physical conditions and provide appropriate notification in accordance with a physical condition.

### BRIEF DESCRIPTION OF THE DRAWINGS

FIG. 1 is a diagram for describing a usage form of a communication terminal;

FIG. 2 is a diagram showing an example of a configuration of the communication terminal;

FIG. 3 is a diagram showing a relationship between evaluation target items, a plurality of intellectual functions, and driving advice items;

FIG. 4 is a flowchart for describing a notification method executed by the communication terminal;

FIG. 5 is a diagram for describing driving risk determination processing and notification processing while driving; and

FIG. 6 is a diagram showing an example of dialog processing.

### DETAILED DESCRIPTION OF THE INVENTION

### 1. Usage Form of Communication Terminal 1

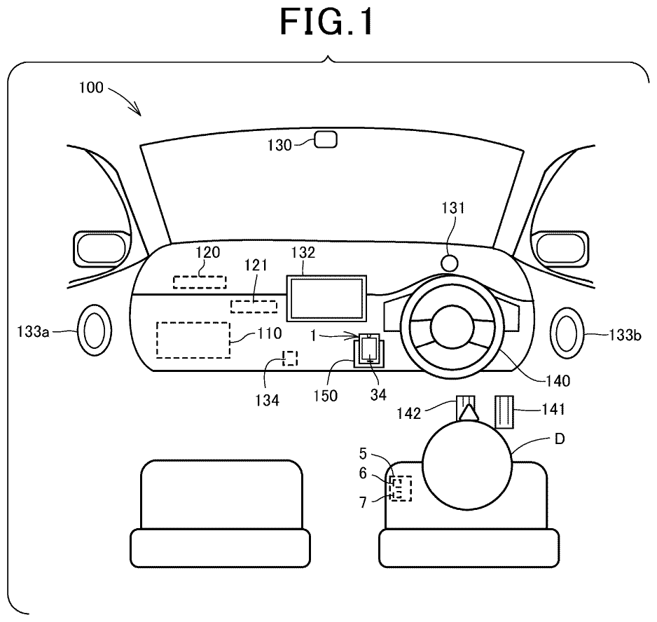

As shown in FIG. 1, a communication terminal 1 is attached via a terminal holder 150 at a position in front of a driver D being a user in a vehicle 100. The communication terminal 1 is a portable device such as a smartphone, a mobile phone, or a tablet terminal, and can be carried by the driver D. The vehicle 100 is a four-wheeled vehicle, and is an example of a target vehicle of the present disclosure. Note that the target vehicle may be a mobile body other than a four-wheeled vehicle, and may be, for example, an aircraft, a motorcycle, or a ship.

A wearable device 5 (computer) is worn by the driver D. The wearable device 5 is a device such as a smartwatch that is worn on a wrist, an arm, the head, or the like. Alternatively, the wearable device 5 is a sensing device attached to smart innerwear to be worn, the skin, inside the body, or the like. The wearable device 5 includes a vitals sensor 6 and a communication unit 7 (transmitter/receiver, circuit).

The vitals sensor 6 is a sensor that continuously measures biological information Db of the driver D at time intervals. This vitals sensor 6 includes a sensor capable of measuring the biological information Db relating to sleep, exercise, and stress of the driver D. For example, the sensor is included in a sensor group capable of measuring information relating to a heart rate, wrist skin temperature being an indicator of body temperature, an electrocardiogram, a blood oxygen level, activity amount, and sleep.

The communication unit 7 is a unit that performs wireless communication with the communication terminal 1, and performs, for example, short-range communication in accordance with a specification such as Bluetooth (R) or ultra-wideband (UWB). The wearable device 5 transmits the measured biological information Db to the communication terminal 1 by using the communication unit 7. Note that information about the communication terminal 1 being a communication destination is preregistered in the communication unit 7.

The vehicle 100 includes an electronic control unit (ECU) 110 that controls an operation of each unit of the vehicle 100, a communication unit 120 (transmitter/receiver, circuit), a navigation apparatus 121 (computer), a front camera 130 that images a front view of the vehicle 100, a driver monitor camera 131 that images the driver D, a display 132, speakers 133a and 133b, an acceleration sensor 134, and the like. The ECU 110 performs communication with the communication terminal 1 via the communication unit 120. The vehicle 100 includes a steering wheel 140, an acceleration pedal 141, a brake pedal 142, and the like as driving operation parts that the driver D operates when driving.

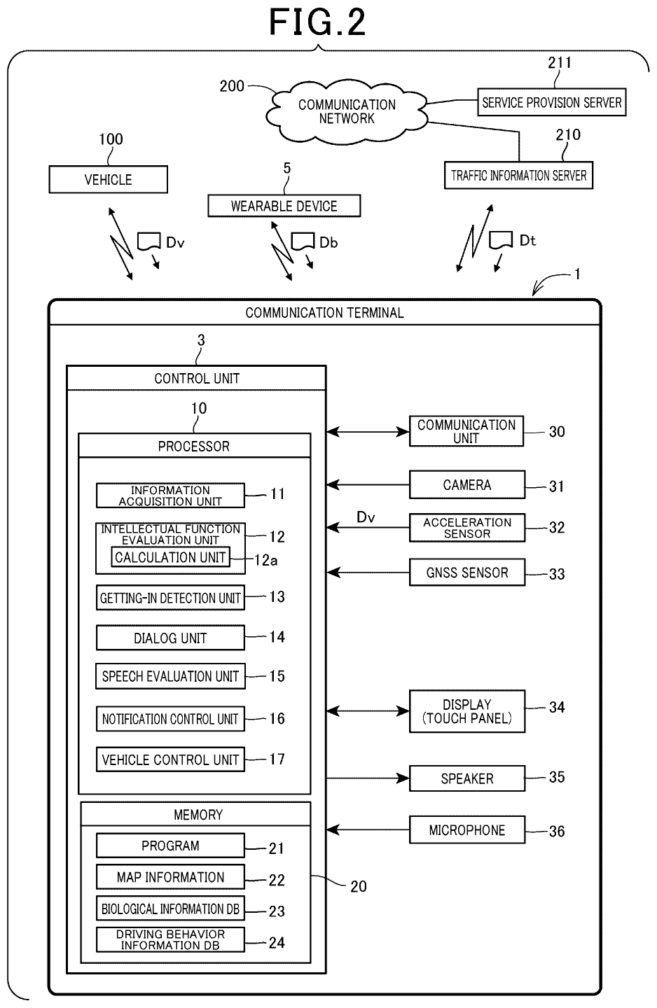

The communication terminal 1 can detect, via an acceleration sensor 32 (see FIG. 2) built in the communication terminal 1, behavior of the vehicle 100 that changes in accordance with operation of the driving operation parts, that is, operation of the steering wheel 140, the acceleration pedal 141, and the brake pedal 142 by the driver D. Note that a notification apparatus 2 may detect the behavior of the vehicle 100 by acquiring a detection result from the acceleration sensor 134 provided in the vehicle 100.

The communication terminal 1 notifies, via a display 34 included in the communication terminal 1, the driver D of notification information based on the biological information Db detected by the wearable device 5 or the like.

Furthermore, the communication terminal 1 includes various functions provided in a typical smartphone or the like, such as a map display function of displaying map information including current position on the display 34, a route guidance function of performing route guidance from a current position to a destination set by the driver D, and a music playback function. Note that the communication terminal 1 may include a function of causing the display 132 of the vehicle 100 to display the advice information, the map information, and the like described above.

### 2. Configuration of Communication Terminal 1

A configuration of the communication terminal 1 will be described with reference to FIG. 2. The communication terminal 1 includes a control unit 3 including a processor 10 and a memory 20, a communication unit 30, a camera 31, the acceleration sensor 32, a global navigation satellite system (GNSS) sensor 33, the display 34, a speaker 35, a microphone 36, and the like.

The communication unit 30 includes a communication module (transmitter/receiver, circuit) that performs wireless communication with external equipment.

The communication unit 30 includes a communication module that performs short-range wireless communication between the vehicle 100, the wearable device 5, and the like in accordance with a specification such as Bluetooth (R) or ultra-wideband (UWB). Furthermore, the communication unit 30 includes a communication module that performs communication between a traffic information server 210, a service provision server 211, and the like via a communication network 200 such as a mobile communication network. Note that information about the vehicle 100 and the wearable device 5 being communication destinations is preregistered at the communication unit 30 side.

The camera 31 can capture the driver D in a state in which the communication terminal 1 is placed in the terminal holder 150 (see FIG. 1). The acceleration sensor 32 detects, for example, six axes (acceleration in each of front-rear, left-right, and up-down axis direction, and angular velocity along each axis). The GNSS sensor 33 detects the current position of the communication terminal 1. A detected position from the GNSS sensor 33 is the current position of the vehicle 100 in the state in which the communication terminal 1 is placed in the terminal holder 150. The driver D can visually confirm the display 34 in the state in which the communication terminal 1 is placed in the terminal holder 150 (see FIG. 1). The display 34 is a touch panel, and the driver D can operate the communication terminal 1 through a touch operation on the display 34.

A program 21 of an application of the communication terminal 1, map information 22 including roads, a biological information database 23 (hereinafter, a biological information DB 23) in which the biological information Db acquired from wearable device 5 is stored in chronological order, and a driving behavior information database 24 (hereinafter, a driving behavior information DB 24) in which driving behavior information Dv to be described below is stored in chronological order are recorded in the memory 20.

The processor 10 is a computer that controls the communication terminal 1, and functions as an information acquisition unit 11, an intellectual function evaluation unit 12, a getting-in detection unit 13, a dialog unit 14, a speech evaluation unit 15, a notification control unit 16, a vehicle control unit 17, and the like by loading and executing the program 21.

The information acquisition unit 11 acquires the biological information Db measured by the wearable device 5 by using the communication unit 30, and records the biological information Db in the memory 20. Note that a measurement date and time of the biological information Db are also stored in the memory 20.

The information acquisition unit 11 acquires information acquired by the camera 31, the acceleration sensor 32, the GNSS sensor 33, and the microphone 36, and stores the information in the memory 20. The information acquired by the camera 31 is, for example, facial image data of the driver D. The facial image data may be used as the biological information Db for identifying a physical condition and the like of the driver D.

The information acquired by the acceleration sensor 32 is information indicating the behavior of the vehicle 100 caused by the operation of the steering wheel 140, the acceleration pedal 141, and the brake pedal 142 by the driver D, that is, the driving behavior information Dv indicating driving behavior of the driver D. The driving behavior information Dv is acquired continuously at time intervals, and is stored in the memory 20.

Note that the present invention is not limited to the information acquisition unit 11 acquiring the driving behavior information Dv by using the acceleration sensor 32 of the communication terminal 1, but the driving behavior information Dv acquired by the acceleration sensor 134 of the vehicle 100 may also be acquired by using short-range communication.

Furthermore, the information acquisition unit 11 can acquire information on the communication network 200 by using the communication unit 30. For example, the information acquisition unit 11 can acquire road traffic information Dt provided by the traffic information server 210 and various provided information provided by the service provision server 211. For example, the provided information from the service provision server 211 is examination information provided by a medical institution (for example, health diagnosis information) about a physical function, a cognitive function, and the like of the driver D; advice provided by a medical institution; or information for identifying an external environment (weather information or the like) of the driver D, the vehicle 100, or the like. The examination information may be used as the biological information Db.

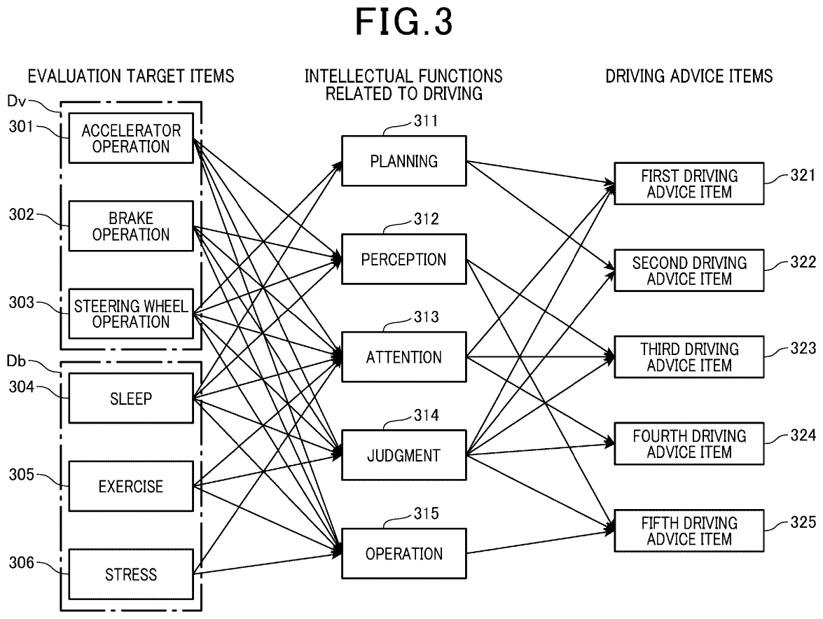

The intellectual function evaluation unit 12 performs processing of evaluating, based on the biological information Db, a plurality of intellectual functions relating to the physical condition of the driver D and related to a driving ability of the driver D. To be more specific, the intellectual function evaluation unit 12 includes a calculation unit 12a that calculates, based on the biological information Db, evaluation values of the plurality of intellectual functions consisting of a planning ability 311, a perception ability 312, an attention ability 313, a judgment ability 314, and an operation ability 315 shown in FIG. 3. The intellectual function evaluation unit 12 evaluates the plurality of intellectual functions based on the calculated evaluation values, and detects whether the physical condition of the driver D is favorable based on these evaluations.

FIG. 3 shows a relationship between evaluation target items, the plurality of intellectual functions to be evaluated by the intellectual function evaluation unit 12 (the planning ability 311, the perception ability 312, the attention ability 313, the judgment ability 314, and the operation ability 315), and driving advice items (a first driving advice item 321 to a fifth driving advice item 325).

Since the biological information Db acquired by the vitals sensor 6 includes information influencing sleep 304, exercise 305, and stress 306 shown in FIG. 3, it is possible to estimate a state of the sleep 304, the exercise 305, and the stress 306 from the biological information Db. Since the driving behavior information Dv includes information about an accelerator operation 301, a brake operation 302, and a steering wheel operation 303 shown in FIG. 3, it is possible to estimate a state of the accelerator operation 301, the brake operation 302, and the steering wheel operation 303 from the driving behavior information Dv.

As shown in FIG. 3, the planning ability 311 is especially related to the sleep 304. The perception ability 312 is related to the sleep 304, the accelerator operation 301, the brake operation 302, and the steering wheel operation 303. The attention ability 313 is related to the sleep 304, the exercise 305, the stress 306, the accelerator operation 301, the brake operation 302, and the steering wheel operation 303. The judgment ability 314 and the operation ability 315 are related to the sleep 304, the exercise 305, the accelerator operation 301, the brake operation 302, and the steering wheel operation 303.

By using these relationships, the intellectual function evaluation unit 12 determines whether these intellectual functions are favorable by: grading the sleep 304, the exercise 305, and the stress 306 of the driver D based on the biological information Db about the driver D; acquiring first physical condition information corresponding to the evaluation value of each intellectual function based on the relationships shown in FIG. 3; and comparing each piece of the first physical condition information with a predetermined threshold value. The intellectual function evaluation unit 12 determines that the physical condition related to the intellectual functions of the driver D (hereinafter denoted as a first physical condition as appropriate) is favorable when a majority (three or more) of the intellectual functions are favorable, and determines that the first physical condition of the driver D is unfavorable when less than a majority (two or less) of the intellectual functions are favorable.

Note that physical condition in the present description is not limited to a narrow meaning, but includes information indicating at least one of physical condition or health with a broad meaning.

Known methods may be applied as appropriate for evaluation processing of the first physical condition, including calculation processing of the evaluation value for each intellectual function. The examination information about the physical function and the cognitive function of the driver D from the service provision server 211 (for example, the health diagnosis information) may be acquired, and the evaluation of each intellectual function, the evaluation of the first physical condition, and the like may be performed based on the biological information Db including this examination information. The intellectual functions may be changed, increased, or decreased as appropriate.

Furthermore, the intellectual function evaluation unit 12 can evaluate the planning ability 311, the perception ability 312, the attention ability 313, the judgment ability 314, and the operation ability 315 based on the driving behavior information Dv, and can evaluate the physical condition of the driver D taking into account both this evaluation and the evaluation based on the biological information Db.

Next, the driving advice items shown in FIG. 3 will be described.

The first driving advice item 321 is a suitable item for giving driving advice when the planning ability 311 or the attention ability 313 is low, and, for example, indicates an operation speed of the acceleration pedal 141 when starting to move.

The second driving advice item 322 is a suitable item for giving driving advice when the planning ability 311 or the judgment ability 314 is low, and, for example, indicates an operation speed of the brake pedal 142 when stopping.

The third driving advice item 323 is a suitable item for giving driving advice when the perception ability 312, the attention ability 313, or the judgment ability 314 is low, and, for example, indicates an operation speed of the steering wheel 140 when negotiating curves, overtaking another vehicle, and the like.

The fourth driving advice item 324 is a suitable item for giving driving advice when the attention ability 313 is low, and, for example, indicates an inter-vehicle distance from a vehicle traveling in front.

The fifth driving advice item 325 is a suitable item for giving driving advice when the perception ability 312, the judgment ability 314, or the operation ability 315 is low, and, for example, indicates left-right drifting of the vehicle 100.

A usage form of these driving advice items will be described below.

Returning to FIG. 2, the getting-in detection unit 13 performs processing of detecting whether the driver D has not gotten in or has gotten in the vehicle, by detecting a timing when the driver D gets in the vehicle 100 (hereinafter, a getting-in timing). The processing of detecting the getting-in timing of the driver D is not particularly limited, but the getting-in detection unit 13, for example, acquires image information from a camera in the vehicle 100 (for example, the driver monitor camera 131) or a camera provided in the vehicle 100 that captures an outside of the vehicle, detects the getting-in timing of the driver D by using image recognition technology. The getting-in detection unit 13 may receive a signal from a weight sensor provided at the driver seat from the vehicle 100, and may detect the getting-in timing of the driver D based on the received signal.

The dialog unit 14 performs dialog processing of having a dialog with the driver D. The dialog processing is processing of performing speech output for dialog encouraging a speech response from the driver D by using the speaker 133a. This dialog processing includes processing of collecting speech information of the driver D via the microphone 36.

In the present embodiment, the dialog processing is executed at the getting-in timing of the driver D detected by the getting-in detection unit 13, or at a timing immediately before or immediately after the getting-in timing. With this, it is possible to acquire the speech information of the driver D when getting in the vehicle 100, immediately before getting in the vehicle 100, or immediately after getting in the vehicle 100, that is, it is possible to acquire the speech information of the driver D immediately before driving the vehicle 100.

The speech evaluation unit 15 performs processing of acquiring physical condition speech information relating to the physical condition of the driver D, by performing speech analysis on the speech information of the driver D acquired through the dialog processing. A content of this processing is not particularly limited, but in the present embodiment, the physical condition speech information indicating an energy level and a mental stability level is acquired by extracting characteristics of the speech and performing arithmetic processing of calculating information indicating the energy level and the mental stability level of the driver D from the extracted characteristics. The speech evaluation unit 15 determines, based on the physical condition speech information, whether a physical condition influenced by the energy level and the mental stability level (denoted as a second physical condition as appropriate) is favorable.

Note that the processing performed by the speech evaluation unit 15 is not limited to the above processing, and can be broadly applied to processing of acquiring information for evaluating the physical condition of the driver D from the speech of the driver D.

The notification control unit 16 generates notification information corresponding to the physical condition of the driver D based on a combination of an evaluation result of the intellectual function evaluation unit 12 and an evaluation result of the speech evaluation unit 15, and displays the generated notification information on the display 34 or emits speech corresponding to the notification information from the speaker 133a.

The vehicle control unit 17 has a control function for the vehicle 100, and more specifically, can perform control related to a driving assistance function of the vehicle 100 by communicating with the vehicle 100 using the communication unit 30. The driving assistance function includes an automatic braking function, a lane keeping assistance function, and adaptive cruise control (ACC).

### 3. Operation of Communication Terminal 1

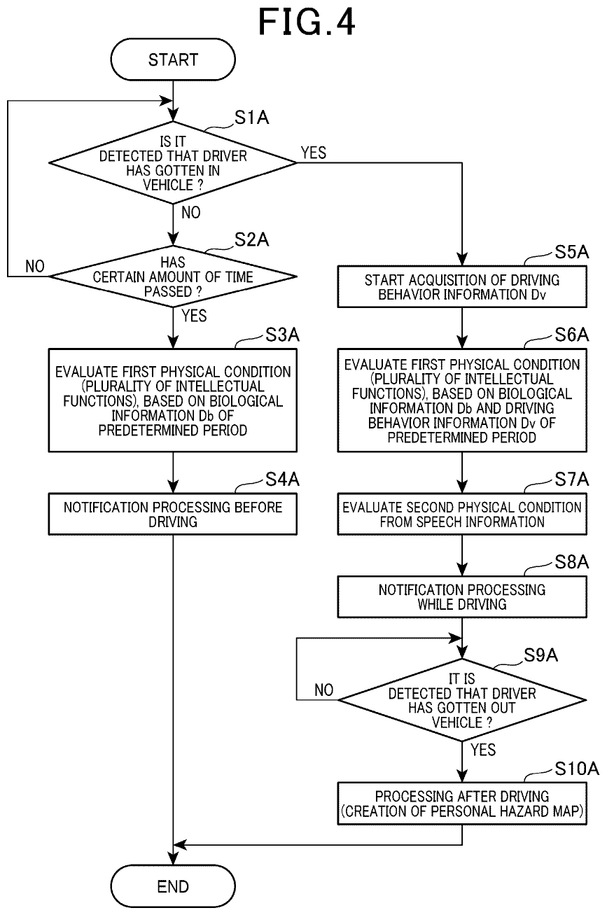

A notification method executed by the communication terminal 1 will be described in accordance with a flowchart shown in FIG. 4. The flowchart shown in FIG. 4 is repeatedly executed at an appropriate interrupt timing when the program 21 of the application is being executed. When the wearable device 5 is present within a short-range wireless communication area of the communication terminal 1, processing of acquiring the biological information Db measured by the wearable device 5 is executed by the information acquisition unit 11.

As shown in FIG. 4, the notification control unit 16 of the communication terminal 1 determines whether the getting-in detection unit 13 has detected that the driver D has gotten in the vehicle 100 (step S1A). When the getting-in detection unit 13 does not detect that the driver D has gotten in the vehicle 100 (NO in step S1A), the notification control unit 16 determines whether a certain amount of time has passed since previously displaying advice on the display 34 (step S2A). When the certain amount of time has not passed (NO in step S2A), the notification control unit 16 transitions to the processing of step S1A.

When the getting-in detection unit 13 does not detect that the driver D has gotten in the vehicle 100 (NO in step S1A) and the certain amount of time passes (YES in step S2A), the notification control unit 16 performs, via the intellectual function evaluation unit 12, processing of acquiring the first physical condition information corresponding to the evaluation value of each of the plurality of intellectual functions based on the biological information Db of a predetermined period in the biological information DB 23 recorded in the memory 20, and evaluating the first physical condition of the driver D based on the first physical condition information (step S3A).

With this, the intellectual function evaluation unit 12 detects whether a majority (three or more) of the intellectual functions is favorable in the predetermined period and detects whether the first physical condition is favorable in accordance with whether a majority of the intellectual functions is favorable for the planning ability 311, the perception ability 312, the attention ability 313, the judgment ability 314, and the operation ability 315.

Next, the notification control unit 16 performs notification processing before driving, based on the evaluation result from the intellectual function evaluation unit 12 (step S4A). For example, when a majority (three or more) of the intellectual functions is favorable, the intellectual function evaluation unit 12 detects that the first physical condition of the driver D is favorable, and the notification control unit 16 displays notification information consisting of suitable advice for health maintenance on the display 34 or emits speech corresponding to the notification information from the speaker 133a.

On the other hand, when less than a majority of the intellectual functions is favorable, the intellectual function evaluation unit 12 detects that the first physical condition of the driver D is unfavorable, and the notification control unit 16 displays notification information consisting of information encouraging exercise, going out, or the like for the purpose of health promotion and/or notification information relating to the intellectual function detected as being the most unfavorable on the display 34, or emits speech corresponding to the notification information from the speaker 133a.

Note that a content of the notification processing before driving may be modified as appropriate and need not be limited to the notification information based on the evaluation result of the plurality of intellectual functions. For example, since the intellectual function evaluation unit 12 can identify the sleep 304, the exercise 305, and the stress 306 based on the biological information Db, the driver D may be notified of notification information relating to the most unfavorable state among the sleep 304, the exercise 305, and the stress 306.

When the getting-in detection unit 13 detects that the driver D has gotten in the vehicle 100 (YES in step S1A), the notification control unit 16 starts acquisition of the driving behavior information Dv (step S5A), and performs, via the intellectual function evaluation unit 12, processing of acquiring the first physical condition information corresponding to the evaluation value of each of the plurality of intellectual functions based on the biological information Db of the predetermined period in the biological information DB 23 and the driving behavior information Dv of the predetermined period in the driving behavior information DB 24, and evaluating the first physical condition of the driver D based on the first physical condition information (step S6A).

With this, the intellectual function evaluation unit 12 detects whether a majority (three or more) of the intellectual functions is favorable in the predetermined period and detects whether the first physical condition is favorable in accordance with whether a majority of the intellectual functions is favorable for the planning ability 311, the perception ability 312, the attention ability 313, the judgment ability 314, and the operation ability 315.

Here, the predetermined period is set to a period in which a relatively long-term physical condition of the driver D (corresponding to the first physical condition) can be identified, and is set to, for example, a period of the past several hours, several days, or several months starting from the current point in time. With this, it is possible to acquire an evaluation result of the relatively long-term physical condition of the driver D. Note that the predetermined period in step S6A may be set longer than the period in step S3A. It is possible to detect the relatively long-term physical condition of the driver D in at least step S6A.

Subsequently, the notification control unit 16 performs driving risk determination processing of determining a driving risk based on the evaluation result of step S6A (step S7A) and performs notification processing while driving based on a determination result of the driving risk determination processing (step S8A). The driving risk determination processing and notification processing while driving will be described below.

The notification control unit 16 determines whether it is detected that the driver D has gotten out of the vehicle 100 after executing the notification processing while driving (step S9A). When it is detected that the driver D has gotten out of the vehicle 100 (YES in step S9A), processing after driving is performed (step S10A). A method of detecting the driver D getting out of the vehicle 100 is not particularly limited, but a method may, for example, be applied of using the front camera 130, a weight sensor at the driver seat, or the like.

The processing after driving in step S10A is processing of creating a hazard map on which locations where inappropriate driving behavior has occurred are recorded, based on the driving behavior information Dv acquired while driving. For example, the information acquisition unit 11 continuously acquires the driving behavior information Dv while the vehicle is traveling, and the notification control unit 16 detects the locations where inappropriate driving behavior such as abrupt braking and left-right drifting has occurred based on the driving behavior information Dv and information about the current position detected by the GNSS sensor 33, and performs the processing of creating a hazard map on which a detection result is recorded. The created hazard map may be data recorded in the map information 22 of the memory 20, or may be data recorded in the memory 20 separate from the map information 22.

When a hazard map is created, it is possible, by using the hazard map while driving, to, for example, via the notification control unit 16, notify the driver D of a location where inappropriate driving behavior has occurred and encourage the driver D to drive safely when traveling nearby this location. Note that the processing after driving is not limited to the processing of creating a hazard map, and appropriate processing may be applied.

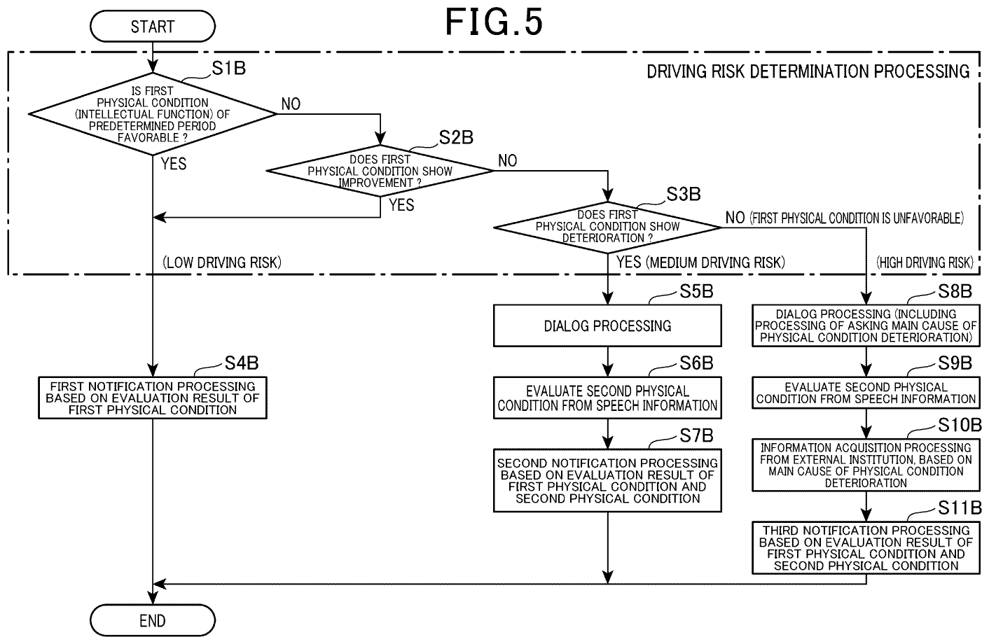

The driving risk determination processing and the notification processing while driving will be described in accordance with a flowchart in FIG. 5.

As shown in FIG. 5, the notification control unit 16 determines, via the intellectual function evaluation unit 12, whether the first physical condition of the predetermined period is favorable (step S1B), whether the first physical condition shows improvement (step S2B), whether the first physical condition shows deterioration (step S3B), and the like.

Note that the notification control unit 16 determines that the first physical condition is favorable when the first physical condition of the predetermined period is continuously favorable, determines that the first physical condition shows improvement when the first physical condition of the predetermined period is gradually changing towards being favorable, determines that the first physical condition shows deterioration when the first physical condition of the predetermined period is gradually changing towards being unfavorable, and determines that the first physical condition is unfavorable when the first physical condition of the predetermined period is continuously unfavorable. This determination method may be set as appropriate.

When the first physical condition is favorable (YES in step S1B) or when the first physical condition shows improvement (YES in step S2B), the notification control unit 16 determines that the driving risk is low (hereinafter, low driving risk) and performs first notification processing based on an evaluation result of the first physical condition (step S4B).

In the first notification processing, the driver D is notified of minimal notification information for a user with a low driving risk (for example, “Safe travels.”). If the physical condition shows improvement, minimal notification information is sufficient, since it is likely that the body can easily handle temporary discomfort.

When the first physical condition shows deterioration (YES in step S3B), the notification control unit 16 determines that the driving risk is medium (hereinafter, medium driving risk) and executes the processing of steps S5B to S7B.

In step S5B, the notification control unit 16 performs the dialog processing via the dialog unit 14.

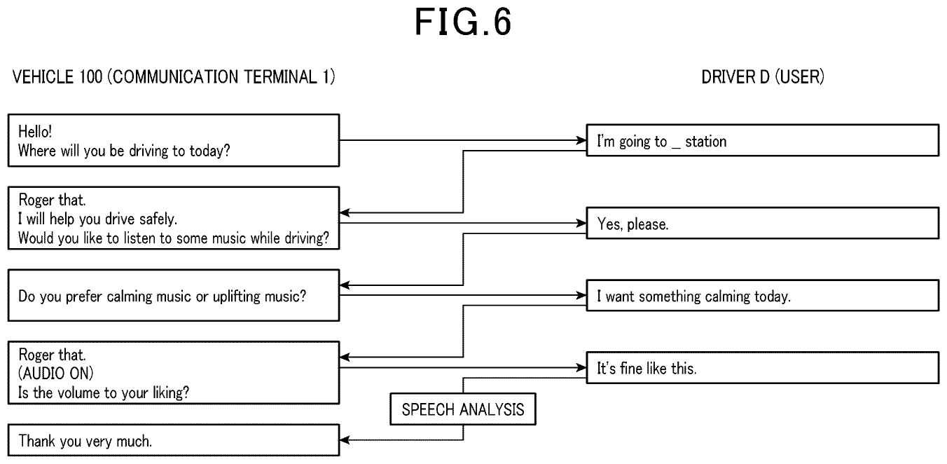

FIG. 6 is a diagram showing an example of the dialog processing.

A left side of FIG. 6 shows notification information emitted by the dialog unit 14 via the speaker 133a. Note that the notification information may be displayed on the display 34. A right side of FIG. 6 shows utterance content produced by the driver D. The speech produced by the driver D is collected by the microphone 36.

A dialog content of the dialog unit 14 may be changed appropriate within the scope of encouraging a speech response from the driver D. In the example of FIG. 6, the dialog content includes a message asking a destination (“Where will you be driving to today?”) and a message asking whether to playback audio and an audio playback preference (“Would you like to listen to music while driving?”, “Do you prefer relaxing music or uplifting music?”, and the like).

Thus, the communication terminal 1 can acquire information relating to the destination, the audio playback, and the audio preference produced by the driver D, by using known speech recognition technology. The communication terminal 1 can start route guidance processing to the destination and audio playback processing, by executing processing corresponding to the acquired information.

Note that when performing the route guidance processing, it is preferable to acquire the road traffic information Dt provided by the traffic information server 210, search for a recommended route by using congestion information and the like included in the road traffic information Dt, or the like.

In step S6B shown in FIG. 5, the notification control unit 16 performs, via the speech evaluation unit 15, speech analysis on the speech information of the driver D acquired through the dialog processing, acquires second physical condition information consisting of physical condition speech information indicating the energy level and the like of the driver D, and evaluates the second physical condition of the driver D based on the second physical condition information (step S6B). With this, it is possible to acquire an evaluation result of the current physical condition of the driver D from the speech, that is, acquire an evaluation result of a relatively short-term physical condition of the driver D.

In step S7B, the notification control unit 16 executes second notification processing based on the evaluation result of the first physical condition and the second physical condition.

The second notification processing is processing, which is different from the first notification processing, of notifying the driver D of information, and is processing of notifying the driver D of suitable notification information when the driving risk is medium. For example, the suitable notification information includes advice relating to a driving operation and advice relating to the physical condition. The driver D is notified of the advice relating to the driving operation by using a driving advice item corresponding to at least the intellectual function with the lowest evaluation from among the driving advice items 321 to 325 shown in FIG. 3.

To give a specific example, when the intellectual function with the lowest evaluation is the operation ability 315, the driver D is notified of notification information including “Be mindful of left-right drifting of the vehicle 100 when driving today.”, since the driving advice item corresponding to the operation ability 315 is the fifth driving advice item 325 (left-right drifting of the vehicle 100).

Regarding the advice relating to the physical condition, the driver D is notified of advice relating to an intellectual function showing deterioration among the plurality of intellectual functions corresponding to the first physical condition (For example, when the attention ability is low, the driver D is notified of advice such as “Your attention ability seems to be low lately. Be careful when driving.”). When the second physical condition is not favorable, the driver D is, for example, notified of notification information including advice such as “Your current physical condition does not seem good. Be mindful to drive safely without straining yourself.”

As shown in FIG. 5, when the first physical condition does not show deterioration, but the first physical condition of the predetermined period is continuously unfavorable (NO in step S3B), the notification control unit 16 determines that the driving risk is high (hereinafter, high driving risk) and executes the processing of steps S8B to S11B.

In step S8B, the notification control unit 16 performs, similar to step S5B, the dialog processing via the dialog unit 14 and performs processing of asking a main cause of physical condition deterioration (for example, a question such as “Please share where you are not feeling well.”). With this, speech information corresponding to the main cause of the physical condition deterioration is acquired.

Subsequently, in step S9B, the notification control unit 16 performs, via the speech evaluation unit 15, the processing of evaluating the physical condition (corresponding to the second physical condition) of the driver D based on the speech information of the driver D acquired through the dialog processing, similar to step S6B.

In step S10B, the notification control unit 16 identifies text corresponding to the main cause of the physical condition deterioration by performing speech recognition on text included in the speech information acquired through the dialog processing of step S8B, and performs information acquisition processing of acquiring advice information corresponding to the physical condition deterioration based on the identified text.

For example, upon recognizing the text relating to physical condition deterioration such as “stomachache” or “headache” from the speech information, the notification control unit 16 acquires advice information corresponding to “stomachache”, “headache”, or the like from a medical institution by making an inquiry to the service provision server 211 via the information acquisition unit 11, based on this text.

In step S11B, the notification control unit 16 executes third notification processing based on the evaluation result of the first physical condition and the second physical condition.

The third notification processing is processing, which is different from the first notification processing and the second notification processing, of notifying the driver D of information, and is processing of notifying the driver D of suitable notification information when the driving risk is high. In the present embodiment, the driver D is notified of notification information including advice relating to the driving operation and advice relating to the physical condition. Regarding the advice relating to the driving operation, the driver D is notified of the notification information including the advice by using the driving advice item corresponding to at least the intellectual function with the lowest evaluation from among the driving advice items 321 to 325 shown in FIG. 3.

Regarding the advice relating to the physical condition, the driver D is notified of the advice information from the medical institution acquired in step S10B. With this, it is possible to notify the driver D of specialized advice, and to perform appropriate notification for the driver D.

Note that when performing the second notification processing and the third notification processing, settings changing processing of the driving assistance function may be executed.

The settings changing processing of the driving assistance function is processing of changing, via the vehicle control unit 17, the driving assistance function of the vehicle 100 to a predetermined setting prioritizing safety. The predetermined setting is, for example, a longer inter-vehicle distance during adaptive cruise control. Note that when performing the first notification processing and the second notification processing after performing the settings changing processing of the driving assistance function, that is, when the physical condition of the driver D has improved, the settings of the driving assistance function may be returned to the pre-changed settings (for example, user-set values).

When performing the third notification processing, the notification processing for a surrounding area may be further executed.

The notification processing for a surrounding area is processing of notifying, by the notification control unit 16 using the communication unit 30, an occupant of another vehicle in a surrounding area of the vehicle 100, a family of the driver D, and the like of surrounding area advice relating to the driving of the driver D, by wirelessly communicating the surrounding area advice. For example, by wirelessly transmitting information corresponding to the surrounding area advice to an in-vehicle system of another vehicle, a mobile terminal carried by a passenger or a family member, or the like, the surrounding area advice is emitted from the in-vehicle system, the mobile terminal (e.g., a smartphone), or the like. With this, it is possible to notify the surrounding area calling for attention, when the physical condition of the driver D is unfavorable. Note that inter-vehicle communication may be used for the communication to the in-vehicle system of the other vehicle or the like.

### 4. Effects of Embodiment

As described above, the communication terminal 1 includes: the information acquisition unit 11 that acquires the biological information Db about the driver D being the user at time intervals; the intellectual function evaluation unit 12 that evaluates the first physical condition indicating the relatively long-term physical condition of the driver D, based on a plurality of pieces of the biological information Db of the predetermined period acquired by the information acquisition unit 11; the dialog unit 14 that performs the speech output encouraging the speech response from the driver D, when detecting that the driver D has gotten in the vehicle 100; the speech evaluation unit 15 that evaluates the second physical condition indicating the relatively short-term physical condition of the driver D, based on the speech information of the driver D as the response to the speech output; and the notification control unit 16 that notifies the driver D of the notification information corresponding to the physical condition of the driver D, based on the combination of the evaluation result from the intellectual function evaluation unit 12 and the evaluation result from the speech evaluation unit 15.

According to this configuration, it is possible to separate chronic physical conditions from temporary physical conditions and provide appropriate notification in accordance with the physical condition. With this, it is possible to realize a healthcare mobility service that maintains the safety and the health of the user, and to further improve traffic safety and contribute to development of a sustainable transport system.

It becomes easier to acquire suitable speech information for the evaluation of the second physical condition and it is possible to expect an improvement in evaluation accuracy, since the relatively short-term second physical condition of the driver D is evaluated based on the speech information of the driver D as the response to the speech output.

Note that the information acquisition unit 11 can be referred to as a biological information acquisition unit, the intellectual function evaluation unit 12 can be referred to as a first physical condition evaluation unit, the dialog unit 14 can be referred to as a speech output unit, and the speech evaluation unit 15 can be referred to as a second physical condition evaluation unit.

The communication terminal 1 includes the information acquisition unit 11 that acquires the driving behavior information Dv. The intellectual function evaluation unit 12 evaluates the first physical condition consisting of the relatively long-term physical condition of the driver D, based on the biological information Db and the driving behavior information Dv. According to this configuration, it becomes easier to notify the driver D of suitable advice, due to a combination of the biological information Db and the driving behavior information Dv.

The notification control unit 16 notifies the driver D of the notification information based on only the evaluation result from the intellectual function evaluation unit 12, when the first physical condition shows improvement. According to this configuration, if the physical condition shows improvement, minimal notification information is sufficient and the driver D less likely feels bothered, since it is likely that the body can easily handle temporary discomfort.

The second physical condition is evaluated by the intellectual function evaluation unit 12 and the speech evaluation unit 15 when the first physical condition shows deterioration, and the notification control unit 16 notifies the driver D of the notification information based on the combination of the evaluation result from the intellectual function evaluation unit 12 and the evaluation result from the speech evaluation unit 15. According to this configuration, it becomes easier to notify the driver D of suitable advice taking into account the second physical condition based on the speech from the user, when the physical condition shows deterioration. It is possible to realize a notification system that is not bothersome to the user, since it is possible to omit the processing of the speech output or the like necessary for acquiring the second physical condition.

Note that in the present embodiment, a case has been described in which the notification control unit 16 evaluates the second physical condition via the intellectual function evaluation unit 12 and the speech evaluation unit 15 when the driver D has gotten in the vehicle 100 and the first physical condition shows deterioration, and notifies the driver D of the notification information based on the combination of the evaluation result from the intellectual function evaluation unit 12 and the evaluation result from the speech evaluation unit 15, but the present invention is not limited thereto. The notification control unit 16 may evaluate the second physical condition via the intellectual function evaluation unit 12 and the speech evaluation unit 15 regardless of whether the driver D has gotten in the vehicle 100 when the first physical condition shows deterioration, and notify the driver D of the notification information based on the combination of the evaluation result from the intellectual function evaluation unit 12 and the evaluation result from the speech evaluation unit 15. With this, it becomes easier to notify the driver D of suitable advice taking into account the second physical condition based on the speech information of the user, when the physical condition of the user shows deterioration.

The notification control unit 16 recognizes the text included in the speech information of the driver D, identifies the main cause of the physical condition deterioration based on the text, and notifies the driver D of the advice information corresponding to the identified main cause, when the first physical condition shows deterioration. According to this configuration, it becomes easier to identify the main deterioration cause from the speech of the user and notify the user of suitable advice taking into account the main deterioration cause, when the physical condition shows deterioration.

In this case, the notification control unit 16 performs the information acquisition processing of acquiring the advice information from the service provision server 211 being an external institution, based on the identified main cause of the physical condition deterioration. According to this configuration, it becomes easier to acquire specialized advice information corresponding to the main deterioration cause, and to notify the user of suitable advice.

Note that the processing of the information acquisition unit 11 is an example of a biological information acquisition step according to the present disclosure. The processing of the intellectual function evaluation unit 12 is an example of a first physical condition evaluation step according to the present disclosure. The processing of the dialog unit 14 is an example of a speech output step according to the present disclosure. The processing of the speech evaluation unit 15 is an example of a speech output step according to the present disclosure. The processing of the notification control unit 16 is an example of a notification control step according to the present disclosure.

The notification information includes the surrounding area advice relating to the driving of the driver D. When performing the third notification processing, the notification control unit 16 performs processing of transmitting the surrounding area advice to an external destination by using wireless communication. According to this configuration, it is possible to notify the surrounding area calling for attention, when the physical condition is unfavorable and the driver D is driving the vehicle 100 while still feeling unwell.

The communication terminal 1 includes the vehicle control unit 17 that changes the driving assistance function of the vehicle 100 driven by the driver D to the predetermined setting prioritizing safety, when performing at least one of the second notification processing or the third notification processing. According to this configuration, it is possible to perform driving assistance prioritizing safety, in other words, to perform driving assistance matched to a user with an unfavorable physical condition, when the physical condition is unfavorable and the driver D is driving the vehicle 100 while still feeling unwell.

### 5. Other Embodiments

The above-described embodiment is merely one embodiment according to the present invention and can be freely modified and applied without departing from the gist of the present invention.

For example, the notification information may include content of the information acquired from the service provision server 211 and indicating the external environment (weather information or the like) of the driver D, the vehicle 100, or the like. The configuration of the communication terminal 1 shown in FIG. 2 is one example and may be modified as appropriate. A configuration realized by software, a configuration realized by hardware, or a configuration realized by combining software and hardware can be broadly applied to the configuration of each unit shown in FIG. 2 and the like. The way of dividing processing units in the flowchart, the processing order thereof, and the like are not limited to the illustrated example, and may be modified as appropriate.

Note that in the flowchart shown in FIG. 4, a case is exemplified in which the first physical condition of the driver D is evaluated in step S3A before the driving, based on only the biological information Db of the predetermined period, but the present invention need not be limited thereto. For example, in step S3A as well, the first physical condition of the driver D may be evaluated based on the biological information Db of the predetermined period and the driving behavior information Dv of the predetermined period, similar to step S6A while driving.

A case has been described in which the notification apparatus of the present invention is realized by the communication terminal 1 being a portable device, but the present invention is not limited thereto. The notification apparatus of the present invention may be realized by the vehicle 100. A case has been described in which the notification apparatus acquires the biological information Db from the wearable device 5, but the notification apparatus itself may have a function of measuring the biological information Db.

A case has been described in which the program 21 for realizing the notification method of the present invention is recorded in the notification apparatus, but the program 21 may be acquired from external equipment via communication. The program 21 may be recorded on a recording medium so as to be readable by a computer. A magnetic recording medium, an optical recording medium, a semiconductor memory device, or the like can be used as the recording medium.

### 6. Configurations Supported by Above Embodiments

The above embodiments are specific examples of the following configurations.

### Configuration 1

A notification apparatus includes: a biological information acquisition unit configured to acquire biological information about a user at time intervals; a first physical condition evaluation unit configured to evaluate a first physical condition indicating a relatively long-term physical condition of the user, based on a plurality of pieces of the biological information of a predetermined period acquired by the biological information acquisition unit; a speech output unit configured to perform speech output encouraging a speech response from the user, when detecting that the user has gotten in a target vehicle; a second physical condition evaluation unit configured to evaluate a second physical condition indicating a relatively short-term physical condition of the user, based on speech information of the user as a response to the speech output; and a notification control unit configured to notify the user of notification information corresponding to a physical condition of the user, based on a combination of an evaluation result from the first physical condition evaluation unit and an evaluation result from the second physical condition evaluation unit.

According to this configuration, it is possible to separate chronic physical conditions from temporary physical conditions and provide appropriate notification in accordance with the physical condition.

### Configuration 2

The notification apparatus according to Configuration 1 further includes an information acquisition unit configured to acquire driving behavior information indicating driving behavior of the user, in which the first physical condition evaluation unit evaluates the first physical condition consisting of the relatively long-term physical condition of the user, based on the biological information and the driving behavior information.

According to this configuration, it becomes easier to notify the user of suitable advice, due to a combination of the biological information and the driving behavior information.

### Configuration 3

The notification apparatus according to Configuration 1 or 2, in which the notification control unit notifies the user of notification information based on only the evaluation result from the first physical condition evaluation unit, when the first physical condition shows improvement.

According to this configuration, if the physical condition shows improvement, minimal notification information is sufficient and the user less likely feels bothered, since it is likely that the body can easily handle temporary discomfort.

### Configuration 4

The notification apparatus according to any one of Configurations 1 to 3, in which the notification control unit evaluates the second physical condition via the speech output unit and the second physical condition evaluation unit regardless of whether the user has gotten in the target vehicle, when the first physical condition shows deterioration, and the notification control unit notifies the user of the notification information, based on the combination of the evaluation result from the first physical condition evaluation unit and the evaluation result from the second physical condition evaluation unit.

According to this configuration, it becomes easier to notify the user of suitable advice taking into account the second physical condition based on the speech from the user, when the physical condition shows deterioration. It is possible to realize a notification system that is not bothersome to the user, since it is possible to omit the processing of the speech output or the like necessary for acquiring the second physical condition.

### Configuration 5

The notification apparatus according to Configuration 4, in which the notification control unit recognizes text included in the speech information of the user, identifies a main cause of physical condition deterioration based on the text, and notifies the user of advice information corresponding to the identified main cause, when the first physical condition shows deterioration.

According to this configuration, it becomes easier to identify the main deterioration cause from the speech of the user and notify the user of suitable advice taking into account the main deterioration cause, when the physical condition shows deterioration.

### Configuration 6

The notification apparatus according to Configuration 5, in which the notification control unit performs information acquisition processing of acquiring the advice information from an external institution, based on the identified main cause of the physical condition deterioration.

According to this configuration, it becomes easier to acquire specialized advice information corresponding to the main deterioration cause, and to notify the user of suitable advice.

### Configuration 7

A notification method executed by a notification apparatus includes: a biological information acquisition step of acquiring biological information about a user at time intervals; a first physical condition evaluation step of evaluating a first physical condition indicating a relatively long-term physical condition of the user, based on a plurality of pieces of the biological information of a predetermined period acquired in the biological information acquisition step; a speech output step of performing speech output encouraging a speech response from the user, when detecting that the user has gotten in a target vehicle; a second physical condition evaluation step of evaluating a second physical condition indicating a relatively short-term physical condition of the user, based on speech information of the user corresponding to the speech output; and a notification control step of notifying the user of notification information corresponding to a physical condition of the user, based on a combination of an evaluation result from the first physical condition evaluation step and an evaluation result from the second physical condition evaluation step.

According to this method, it is possible to separate chronic physical conditions from temporary physical conditions and provide appropriate notification in accordance with the physical condition.

### Configuration 8

A non-transitory computer readable recording medium recording a program causes a notification apparatus controlled by a computer to function as: a biological information acquisition unit configured to acquire biological information about a user at time intervals; a first physical condition evaluation unit configured to evaluate a first physical condition indicating a relatively long-term physical condition of the user, based on a plurality of pieces of the biological information of a predetermined period acquired by the biological information acquisition unit; a speech output unit configured to perform speech output encouraging a speech response from the user, when detecting that the user has gotten in a target vehicle; a second physical condition evaluation unit configured to evaluate a second physical condition indicating a relatively short-term physical condition of the user, based on speech information of the user as a response to the speech output; and a notification control unit configured to notify the user of notification information corresponding to a physical condition of the user, based on a combination of an evaluation result from the first physical condition evaluation unit and an evaluation result from the second physical condition evaluation unit.

According to this recording medium, it is possible to separate chronic physical conditions from temporary physical conditions and provide appropriate notification in accordance with the physical condition.

### REFERENCE SIGNS LIST

1 communication terminal
5 wearable device
6 vitals sensor
7 communication unit
10 processor
11 information acquisition unit
12 intellectual function evaluation unit
13 getting-in detection unit
14 dialog unit (speech acquisition unit)
15 speech evaluation unit
16 notification control unit
17 vehicle control unit
20 memory
21 program
22 map information
23 biological information database
24 driving behavior information database
30 communication unit
31 camera
32 acceleration sensor
33 GNSS sensor
34 display
35 speaker
36 microphone
100 vehicle (target vehicle)
110 ECU
121 navigation apparatus
130 front camera
131 driver monitor camera
132 display
133a, 133b speaker
150 terminal holder
160 wearable device
200 communication network
210 traffic information server
211 service provision server
D driver
Db biological information
Dv driving behavior information

1. A notification apparatus, comprising:
a biological information acquisition unit configured to acquire biological information about a user at time intervals;
a first physical condition evaluation unit configured to evaluate a first physical condition indicating a relatively long-term physical condition of the user, based on a plurality of pieces of the biological information of a predetermined period acquired by the biological information acquisition unit;
a speech output unit configured to perform speech output encouraging a speech response from the user, when that the user has gotten in a target vehicle is detected;
a second physical condition evaluation unit configured to evaluate a second physical condition indicating a relatively short-term physical condition of the user, based on speech information of the user as a response to the speech output; and
a notification control unit configured to notify the user of notification information corresponding to a physical condition of the user, based on a combination of an evaluation result from the first physical condition evaluation unit and an evaluation result from the second physical condition evaluation unit.

2. The notification apparatus according to claim 1, further comprising an information acquisition unit configured to acquire driving behavior information indicating driving behavior of the user, wherein
the first physical condition evaluation unit evaluates the first physical condition consisting of the relatively long-term physical condition of the user, based on the biological information and the driving behavior information.

3. The notification apparatus according to claim 1, wherein the notification control unit notifies the user of notification information based on only the evaluation result from the first physical condition evaluation unit, when the first physical condition shows improvement.

4. The notification apparatus according to claim 1, wherein
the notification control unit evaluates the second physical condition via the speech output unit and the second physical condition evaluation unit regardless of whether the user has gotten in the target vehicle, when the first physical condition shows deterioration, and
the notification control unit notifies the user of the notification information, based on the combination of the evaluation result from the first physical condition evaluation unit and the evaluation result from the second physical condition evaluation unit.

5. The notification apparatus according to claim 4, wherein the notification control unit recognizes text included in the speech information of the user, identifies a main cause of physical condition deterioration based on the text, and notifies the user of advice information corresponding to the identified main cause, when the first physical condition shows deterioration.

6. The notification apparatus according to claim 5, wherein the notification control unit performs information acquisition processing of acquiring the advice information from an external institution, based on the identified main cause of the physical condition deterioration.

7. A notification method executed by a notification apparatus, comprising:
a biological information acquisition step of acquiring biological information about a user at time intervals;
a first physical condition evaluation step of evaluating a first physical condition indicating a relatively long-term physical condition of the user, based on a plurality of pieces of the biological information of a predetermined period acquired in the biological information acquisition step;
a speech output step of performing speech output encouraging a speech response from the user, when that the user has gotten in a target vehicle is detected;
a second physical condition evaluation step of evaluating a second physical condition indicating a relatively short-term physical condition of the user, based on speech information of the user corresponding to the speech output; and
a notification control step of notifying the user of notification information corresponding to a physical condition of the user, based on a combination of an evaluation result from the first physical condition evaluation step and an evaluation result from the second physical condition evaluation step.

8. A non-transitory computer readable recording medium recording a program for causing a notification apparatus controlled by a computer to function as:
a biological information acquisition unit configured to acquire biological information about a user at time intervals;
a first physical condition evaluation unit configured to evaluate a first physical condition indicating a relatively long-term physical condition of the user, based on a plurality of pieces of the biological information of a predetermined period acquired by the biological information acquisition unit;
a speech output unit configured to perform speech output encouraging a speech response from the user, when that the user has gotten in a target vehicle is detected;
a second physical condition evaluation unit configured to evaluate a second physical condition indicating a relatively short-term physical condition of the user, based on speech information of the user as a response to the speech output; and
a notification control unit configured to notify the user of notification information corresponding to a physical condition of the user, based on a combination of an evaluation result from the first physical condition evaluation unit and an evaluation result from the second physical condition evaluation unit.
